(19) United States
(12) Patent Application Publication
(10) Pub. No.: US 2026/0315091 A1
(43) Pub. Date: Oct. 15, 2026

(54) RECIPROCATING CATHETER MOTION USING ONLY PORTION OF CAM PROFILE

(71) Applicant: KONINKLIJKE PHILIPS N.V., EINDHOVEN (NL)

(72) Inventors: Jeffrey James SHIMON, COLORADO SPRINGS, CO (US); David HILLS, PALMER LAKE, CO (US)

(21) Appl. No.: 19/106,464

(22) PCT Filed: Aug. 22, 2023
(86) PCT No.: PCT/EP2023/072977

§ 371 Date: Feb. 25, 2025

Related U.S. Application Data
(60) Provisional application No. 63/401,762, filed on Aug. 29, 2022.

(51) Int. Cl. A61B 17/32; A61B 17/00

(52) U.S. Cl. CPC A61B 17/32002; A61B 2017/00243; A61B 2017/00292; A61B 2017/00398; A61B 2017/00734; A61B 2017/320024; A61B 2017/320028

(57) ABSTRACT

An intraluminal cutting device is provided. The device includes a flexible elongate member configured to be positioned within a body lumen of a patient and a cutting tip assembly positioned at a distal portion of the flexible elongate member, wherein the cutting tip assembly comprises a cam pin, a cam slot, and a blade configured to cut tissue associated with the body lumen. The cam pin is configured to move within the cam slot along a cam path, resulting in rotation and translation of the blade. The cam pin is configured to oscillate along only a portion of the cam path.

### TECHNICAL FIELD

The subject matter described herein relates to a system for controlling the motion of the cutting tip of a cutting catheter. In particular, the present disclosure describes aspects related to reciprocating motion of the cutting tip. This system has particular but not exclusive utility for removal of implanted leads from a body lumen of a patient.

### BACKGROUND

Surgically implanted cardiac pacing systems, such as pacemakers and defibrillators, play an important role in the treatment of heart disease. Pacemakers treat slow heart rhythms by increasing the heart rate or by coordinating the heart's contraction for some heart failure patients. Implantable cardioverter-defibrillators stop dangerous rapid heart rhythms by delivering an electric shock. Cardiac pacing systems typically include a timing device and a lead, which are placed inside the body of a patient. One part of the system is the pulse generator containing electric circuits and a battery, usually placed under the skin on the chest wall beneath the collarbone. To replace the battery, the pulse generator must be changed by a simple surgical procedure every 5 to 10 years. Another part of the system includes the wires, or leads, which run between the pulse generator and the heart. In a pacemaker, these leads allow the device to increase the heart rate by delivering small timed bursts (e.g., 60 bursts) of electric energy to make the heart beat faster. In a defibrillator, the lead has special coils to allow the device to deliver a high-energy shock and convert potentially dangerous rapid rhythms (ventricular tachycardia or fibrillation) back to a normal rhythm. Additionally, the leads may transmit information about the heart's electrical activity to the pacemaker.

For both of these functions, leads must be in contact with heart tissue. Most leads pass through a vein under the collarbone that connects to the right side of the heart (right atrium and right ventricle). In some cases, a lead is inserted through a vein and guided into a heart chamber where it is attached to the heart. In other instances, a lead is attached to the outside of the heart. To remain attached to the heart muscle, most leads have a fixation mechanism, such as a small screw and/or hooks at the end. Within a relatively short time after a lead is implanted into the body, the body's natural healing process forms scar tissue along the lead and possibly at its tip, thereby fastening it even more securely in the patient's body. Leads usually last longer than device batteries, so leads are simply reconnected to each new pulse generator (battery) at the time of replacement. Although leads are designed to be implanted permanently in the body, occasionally these leads must be removed, or extracted. Leads may be removed from patients for numerous reasons, including but not limited to, infections, lead age, and lead malfunction.

Removal or extraction of the lead may be difficult. As mentioned above, the body's natural healing process forms scar tissue over and along the lead, and possibly at its tip, thereby encasing at least a portion of the lead and fastening it even more securely in the patient's body. In addition, the lead and/or tissue may become attached to the vasculature wall. Both results may, therefore, increase the difficulty of removing the leads from the patient's vasculature.

A variety of tools have been developed to make lead extraction safer and more successful. A mechanical device to extract leads may include one or more flexible tubes called sheaths that passes over the lead and/or the surrounding tissue. One of the sheaths may include a tip having a dilator, a separator and/or a cutting blade, such that upon advancement, the tip cuts or dilates the scar tissue to separate the scar tissue from other scar tissue, including the scar tissue surrounding the lead. In some cases, the tip (and sheath) may also separate the tissue itself from the lead. Once the lead is separated from the surrounding tissue and/or the surrounding tissue is separated from the remaining scar tissue, the lead may be inserted into a hollow lumen of the sheath for removal and/or be removed from the patient's vasculature using some other mechanical devices, such as mechanical traction devices.

The information included in this Background section of the specification, including any references cited herein and any description or discussion thereof, is included for technical reference purposes only and is not to be regarded as subject matter by which the scope of the disclosure is to be bound.

### SUMMARY

Disclosed is a system for controlling motion of the blade of a cutting catheter. Longitudinal translation of the blade is controlled by a cam path, cam channel, or cam slot in the cutting tip, where rotation of the cutting tip causes the cam channel to move relative to a fixed guide pin, thus translating the cutting tip in a longitudinally distal or proximal direction as it rotates. Oscillating or continuous rotation of the cutting tip can thus produce reciprocating axial/longitudinal motion of the blade. Depending on the shape of the cam channel and/or the portion(s) of the cam channel selected for oscillation at a given time, cutting motions of the blade may be primarily rotational, or primarily axial/longitudinal, or combinations thereof. Furthermore, the blade can also be oscillated while it is shielded or unshielded. Aspects of the present disclosure include generating various vibration modes that may be useful in dilating or separating scar tissue, separating a lead from scar tissue, moving the lead or the separated scar tissue into or through the lumen of the cutting catheter, and/or facilitating movement of lead relative to the cutting sheath. The present disclosure provides systems, devices, and methods for controlling different cutting and vibration modes of the cutting catheter, including but not limited to a rotational mode, an impact mode, and a dithering mode.

One general aspect includes an intraluminal cutting device. The intraluminal cutting device includes a flexible elongate member configured to be positioned within a body lumen of a patient, where the flexible elongate member includes a proximal portion and a distal portion; and a cutting tip assembly positioned at the distal portion of the flexible elongate member, where the cutting tip assembly includes a cam pin, a cam slot, and a blade configured to cut tissue associated with the body lumen, where the cam pin is configured to move within the cam slot along a cam path, where the cam pin is configured to oscillate along only a portion of the cam path. Other aspects include corresponding computer systems, apparatus, and computer programs recorded on one or more computer storage devices, each configured to perform the actions of the methods.

Implementations may include one or more of the following features. In some aspects, in a first mode of operation, the cam pin is configured to move along an entirety of the cam path, and where the cam pin oscillating along only the portion of the cam path is associated with a second mode of operation. In some aspects, in the first mode of operation, the blade is configured to provide a first type of cutting of the tissue, and in the second mode of operation, the blade is configured to provide a second type of cutting of the tissue. In some aspects, the first type of cutting of the tissue is associated with rotational movement of the blade, and the second type of cutting of the tissue is associated with longitudinal movement of the blade. In some aspects, the cutting tip assembly is coupled to the flexible elongate member, where, in the second mode of operation, the cam pin oscillating along only the portion of the cam path is associated with the flexible elongate member oscillating. In some aspects, the second mode of operation is associated with rotational movement of the flexible elongate member. In some aspects, the cam pin reciprocating along only the portion of the cam path is associated with a third mode of operation. In some aspects, the motor is configured to control the cam pin to oscillate along only a portion of the cam path. In some aspects, the processor circuit is configured to control the motor. In some aspects, the battery is configured to power the motor and the processor circuit. In some aspects, the cam pin oscillating along only the portion of the cam path is associated with a mode of operation, where the processor circuit is configured to receive a user input associated with the mode of operation, and where the processor circuit is configured to control the motor in response to the user input. In some aspects, the cam pin oscillating along only the portion of the cam path is associated with a mode of operation, where the processor circuit is configured to determine a duration for the mode of operation, and where the processor circuit is configured to control the motor in response to determining the duration. In some aspects, the portion of the cam path is associated with a first region of the cam path and a second region of the cam path. In some aspects, a first region of the cam path and a second region of the cam path are opposite to one another along the cam path. In some aspects, the processor circuit is configured to control the motor such that the cam pin alternates between: oscillating along the first region of the cam path; and oscillating along the second region of the cam path. Implementations of the described techniques may include hardware, a method or process, or computer software on a computer-accessible medium.

One general aspect includes an intraluminal cutting device. The intraluminal cutting device includes a flexible elongate member configured to be positioned within a body lumen of a patient, where the flexible elongate member includes a proximal portion, a distal portion, and a lumen configured to receive a medical device; a handle positioned at the proximal portion of the flexible elongate member, where the handle includes a motor, a processor circuit, and a battery; and a cutting tip assembly coupled to the flexible elongate member, where the cutting tip assembly includes a cam pin, a cam slot, and a blade configured to cut tissue associated with the body lumen, where the processor circuit is configured to control the motor to cause the cam pin to move within the cam slot along a cam path, where the cam pin is configured to oscillate along only a portion of the cam path such that at least one of: the blade is configured to provide repeated longitudinal contact with the tissue; or the flexible elongate member is configured to provide repeated movement relative to the medical device within the lumen of the flexible elongate member. Other aspects include corresponding computer systems, apparatus, and computer programs recorded on one or more computer storage devices, each configured to perform the actions of the methods.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of the cutting blade motion control system, as defined in the claims, is provided in the following written description of various aspects of the disclosure and illustrated in the accompanying drawings.

### BRIEF DESCRIPTION OF THE DRAWINGS

Illustrative aspects of the present disclosure will be described with reference to the accompanying drawings, of which:

FIG. 1 is a diagrammatic schematic view of an intraluminal cutting device or surgical device, such as a cutting catheter device, according to aspects of the present disclosure.

FIG. 2 is a side perspective view of an example surgical device, according to aspects of the present disclosure.

FIG. 3 is a side cross-sectional view of a distal portion of an example sheath assembly, according to aspects of the present disclosure.

FIG. 4 is a perspective side view of the distal end of an example sheath assembly, according to aspects of the present disclosure.

FIG. 5 is a perspective side view of the distal end of an example sheath assembly, according to aspects of the present disclosure.

FIG. 6 is a diagrammatic view of a surgical device that has been introduced into a body lumen of a patient to remove an implanted lead, according to aspects of the present disclosure.

FIG. 7 is a side cross-sectional view of an example sheath assembly removing a lead from a body lumen, according to aspects of the present disclosure.

FIG. 8 is a side front perspective view of an example cutting tip, according to aspects of the present disclosure.

FIG. 9 is a schematic cross-sectional view of the reciprocating motion of a cutting tip of the distal portion of an example sheath assembly, according to aspects of the present disclosure.

FIG. 10 is a schematic cross-sectional view of the reciprocating motion of a cutting tip of the distal portion of the example sheath assembly of FIG. 9, according to aspects of the present disclosure.

FIG. 11 is a schematic cross-sectional view of the reciprocating motion of a cutting tip of the distal portion of the example sheath assembly of FIG. 10, according to aspects of the present disclosure.

FIG. 12 is a schematic representation of a guide pin moving rightward (e.g., clockwise as seen looking proximally from a distal end of the cutting tip) in an example cam channel, according to aspects of the present disclosure.

FIG. 13 is a schematic representation of a guide pin moving leftward (e.g., counterclockwise as seen looking proximally from a distal end of the cutting tip) in the example cam channel of FIG. 12, according to aspects of the present disclosure.

FIG. 14 is a schematic side cross-sectional view of the reciprocating motion of a cutting tip, according to aspects of the present disclosure.

FIG. 15 is a schematic front cross-sectional view of the reciprocating motion of a cutting tip, according to aspects of the present disclosure.

FIG. 16 is a schematic representation of a guide pin oscillating leftward and rightward in an example cam channel, according to aspects of the present disclosure.

FIG. 17 is a schematic representation of a guide pin oscillating leftward and rightward in an example cam channel, according to aspects of the present disclosure.

FIG. 18 is a schematic representation of a guide pin oscillating upward and downward in a steeply sloped cam channel, according to aspects of the present disclosure.

FIG. 19 is a schematic side cross-sectional view of the reciprocating motion of a cutting tip, according to aspects of the present disclosure.

FIG. 20 is a schematic front cross-sectional view of the reciprocating motion of a cutting tip, according to aspects of the present disclosure.

FIG. 21 is a schematic representation of a guide pin oscillating leftward and rightward in an example cam channel, according to aspects of the present disclosure.

FIG. 22 is a schematic representation of a guide pin oscillating leftward and rightward in an example cam channel, according to aspects of the present disclosure.

FIG. 23 shows a slope of the cam path that may be selected for the dithering mode, according to aspects of the present disclosure.

FIG. 24 shows a slope of the cam path that may be selected for the dithering mode, according to aspects of the present disclosure.

FIG. 25 shows a slope of the cam path that may be selected for the dithering mode, according to aspects of the present disclosure.

FIG. 26 shows a slope of the cam path that may be selected for the dithering mode, according to aspects of the present disclosure

FIG. 27 is a schematic side cross-sectional view of the reciprocating motion of a cutting tip, according to aspects of the present disclosure.

FIG. 28 is a schematic front cross-sectional view of the reciprocating motion of a cutting tip, according to aspects of the present disclosure.

FIG. 29 illustrates a flow diagram for an example method for transition from a rotational mode to an impact mode, according to aspects of the present disclosure.

FIG. 30 illustrates a flow diagram for an example method for operation in a rotational mode, according to aspects of the present disclosure.

FIG. 31 illustrates a flow diagram for an example method operation in an impact mode, according to aspects of the present disclosure.

FIG. 32 illustrates a flow diagram for an example method for transition from a rotational mode to a mobility or dithering mode, according to aspects of the present disclosure.

FIG. 33 illustrates a flow diagram for an example method for operation in a mobility or dithering mode, with time limit, according to aspects of the present disclosure.

FIG. 34 illustrates a flow diagram for an example method for switching between a rotational mode and a default mobility or dithering mode, with time limits, according to aspects of the present disclosure.

FIG. 35 illustrates a flow diagram for an example method for default operation in a mobility or dithering mode with time limit, according to aspects of the present disclosure.

FIG. 36 is a schematic diagram of a processor circuit, according to aspects of the present disclosure.

### DETAILED DESCRIPTION

In some cases, a surgical sheath or catheter device may have a trigger mechanism for extending the blade from the distal end of the sheath. Controlling the amount of extension and retraction of the blade within a patient's vasculature may be critical, particularly when the sheath and blade negotiate tortuous paths that exist in certain vascular or physiological environments and/or when the blade is attempting to cut and/or separate tough surrounding tissue. Furthermore, in certain cases, using such mechanical devices for lead removal may require more meticulous control, such as when the leads are located in, and/or attached to, a structurally weak portion of the vasculature. For instance, typical leads in a human may pass through the innominate vein, past the superior vena cava (“SVC”), and into the right atrium of the heart. Tissue growth occurring along the SVC and other locations along the innominate vein may increase the risk and difficulty in extracting the leads from such locations, particularly when the vein walls are thin and the surrounding tissue is notably fibrous.

The present disclosure provides systems, devices, and methods for controlling different motions of the cutting blade of a cutting catheter. Longitudinal translation of the catheter's cutting blade is controlled by a cam path, cam channel, or cam slot in the cutting tip, where rotation of the cutting tip causes the cam channel to move relative to a fixed guide pin, thus translating the cutting tip in a longitudinally distal or proximal direction as it rotates. Oscillating or continuous rotation of the cutting tip can thus produce reciprocating or oscillating axial/longitudinal motion of the cylindrical blade at the cutting tip's distal edge. Depending on the shape of the cam channel and/or the portion(s) of the cam channel selected for oscillation at a given time, cutting motions of the blade may be primarily rotational, or primarily axial/longitudinal, or combinations thereof. In an example, the portion of the cam path can be between 1% and 75%, between 1% and 50%, between 1% and 25%, between 1% and 10%, between 1% and 5% of a total length of the cam path. Oscillation between two particular points along the cam channel may be selected and controlled by hardware, software, firmware, or combinations thereof, to produce the cutting modes disclosed herein. To enable selection of movement in a particular part of the cam channel, it may be desirable to register the rotational position of the shaft. This may be done for example with a relative rotary encoder, absolute rotary encoder, optical homing sensor, etc.

Furthermore, the blade can also be oscillated or reciprocated while it is shielded within the catheter (e.g., while the blade is not exposed) or unshielded (e.g., while the blade is exposed. Aspects of the present disclosure include generating various desirable vibration modes that may be useful in dilating or separating scar tissue, separating a lead from scar tissue, and/or moving the lead or the separated scar tissue into or through the central lumen of the cutting catheter. The present disclosure provides systems, devices, and methods for controlling different cutting and vibration modes of the blade of the cutting catheter, including but not limited to a rotational mode, an impact mode, and a dithering mode.

The present disclosure can relate to any single- or multi-lumen medical device with a flexible working length capable of traversing venous anatomy and intracardiac leads, that includes a cutting mechanism that can be used to remove plaques or lesions bound onto and around pacemaker lead wires. The cutting mechanism is constrained by a cam profile allowing continuous rotation, or combination, rectilinear/rotational motion; by using specific motor control techniques. High frequency linear motion can cause impacting stress on adjacent tissues (e.g., scar tissue, calcified scar tissue, etc.), which may be beneficial. Such reciprocating rectilinear motion, controlled by a cam path, may be used for intraluminal, intravascular, and intracardiac, excision, biopsy, Targeted Lead Extraction (TLE), or other applications where a cutting catheter may be used. Such applications, even where not described herein, fall within the scope of the present disclosure.

Current projects in-development use electrically motorized, microprocessor control to actuate the mechanical dilation (cutting) mechanism. The systems, devices, and methods disclosed herein use the cam path of the cutting mechanism, along with the microprocessor controlled motor movement, to establish a reciprocating linear movement of the cutting mechanism. This is distinct form the existing movement profile which focuses on rotation, with a relatively small linear output. In some instances, the systems and methods disclosed herein also increase the relative speed of linear motion of the blade. For example, current systems may extend the blade approximately 0.015″, over a relatively long rotational path, whereas the impact mode disclosed herein could exhibit the same 0.015″ linear excursion over a shorter rotational path and thus a higher oscillation rate and higher linear speed.

The present disclosure utilizes a cam profile intended for quickly exposing the blade from a retracted position, then rotating the blade across approximately 60% of a rotation. Because this operation mode relies on the rotational motion of the device, only a small amount of the available power is issued linearly along the main axis of the shaft. This presents several disadvantages.

For example, the exposed rotating cutting blades may create a relatively large area of damage to the patient. The disclosed implementation methodology can reduce the area of damage, if exposed blades contact patient tissues, since the motion may be predominantly axial. Furthermore, existing designs may include a scalloped cutting mechanism shape, which can cut into intracardiac device (ICD) leads, preventing clinical progress. The systems, devices, and methods disclosed herein render this scalloped shape inconsequential to the target or adjacent leads, due to the axial motion, which may increase the safety and efficacy of ICD lead removal procedures. Repetitive axial motion may also shock or impact hard tissues during targeted lead extraction procedures, expediting their traversal and subsequent removal. This feature is not available in existing designs. Advantageously, the physical elements required for certain implementations of the present disclosure are already present within some existing cutting catheter devices, such that the present disclosure may be implemented exclusively or primarily through software changes. These elements include: a cam profile guided cutting mechanism, a reversible motor, and a programmable motor controller (processor). In other cases, implementation of the present disclosure may require new devices with customized features.

The present disclosure substantially aids a clinician in removing an implanted lead from a body lumen of a patient, by providing an assortment of different cutting and/or vibration modes that have different effects on the lead and the scar tissue surrounding it. The system may include cutting tips with specialized cam paths, or may include software or hardware modifications to enable currently existing cutting tips (whether commercially available or otherwise) to exhibit specialized movement profiles. Implemented on a cutting catheter in communication with a handle assembly, the system disclosed herein may provide both time savings and an improvement in the safety and precision of lead removal procedures. This improved lead removal workflow transforms a slow, painstaking process into one where the functionality of the cutting catheter can be changed to suit the needs of different locations along the lead, without the normally routine need to switch to a different cutting catheter. This unconventional approach improves the functioning of the lead removal system, by allowing a single cutting catheter to operate in multiple different modes, including impact and dithering modes that may not be available in current devices at all. The devices, systems, and methods described herein can include one or more features described in U.S. Pat. No. 10,314,615, filed Feb. 12, 2018, and/or U.S. Pat. No. 9,980,743, filed Aug. 26, 2016, both of which are hereby incorporated by reference in their entirety as though fully set forth herein.

The system disclosed herein may be implemented as a set of logical branches and mathematical operations, whose outputs are viewable on a display, and operated by a control process executing on a processor that accepts user inputs from a trigger, touchscreen interface, or other user interface, and that is in communication with one or more motors controlling rotation of the cutting tip. In that regard, the control process performs certain specific operations in response to different inputs or selections made by a user at different times. Certain structures, functions, and operations of the processor, display, sensors, and user input systems are known in the art, while others are recited herein to enable novel features or aspects of the present disclosure with particularity.

These descriptions are provided for exemplary purposes only, and should not be considered to limit the scope of the disclosure. Certain features may be added, removed, or modified without departing from the spirit of the claimed subject matter.

For the purposes of promoting an understanding of the principles of the present disclosure, reference will now be made to the aspects illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

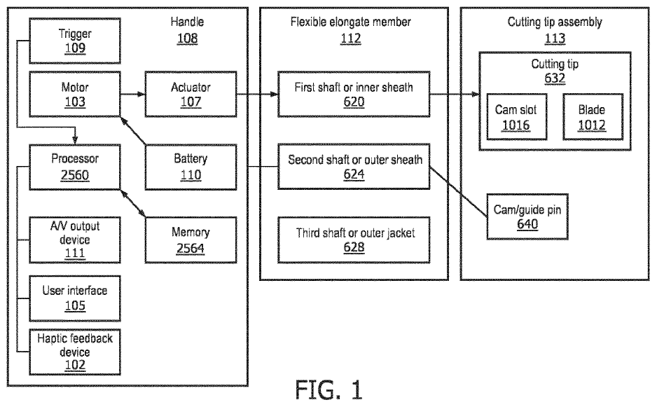

FIG. 1 is a diagrammatic schematic view of an intraluminal cutting device 106 or surgical device 106, such as a cutting catheter device, according to aspects of the present disclosure. The intraluminal cutting device or surgical device 106 includes a handle 108, a flexible elongate member or sheath assembly 112, and a cutting tip assembly 113.

The handle 108 may include a trigger 109, one or more motors 103, an actuator 107, a processor 2560, a battery 110, an audiovisual (A/V) output device 111 (e.g., a display screen, a set of indicator lights, etc.), and a user interface 105 (e.g., a touchscreen, one or more buttons, switches, dials, etc.).

The flexible elongate member or sheath assembly 112 may include a rotatable first shaft or flexible inner sheath 620 coupled to the actuator 107, a fixed second shaft or flexible outer sheath 624 that surrounds the flexible inner sheath 620, and a translatable third shaft or outer jacket 628 that surrounds the flexible outer sheath 624.

In an example, the actuator 107 be or include a power train assembly with one or more gears coupling the 103 motor to the first (inner) shaft or flexible inner sheath 620. A press of the trigger 109 may send a signal to the processor 2560, which then activates the motor 103, which is powered by the battery 110 and transmits rotational motion through the actuator 107 to the flexible inner sheath 620, which then rotates along with the actuator. The flexible outer sheath 624 may be fixed to the handle 108, such that it remains fixed while the flexible inner sheath 620 rotates within it.

The cutting tip assembly 113 may for example include a cutting tip 632 with a cam slot 1016 and blade 1012. The cutting tip 632 is fixedly attached to the flexible inner shaft 620, such that rotation of the flexible inner shaft 620 causes rotation of the cutting tip 632. A cam pin, guide pin, or cam guide pin 640 is fixedly attached to the flexible outer shaft 624, but fits within the cam slot 1016, such that rotation of the cutting tip 632 can also drive axial or longitudinal motion of the cutting tip, as described below.

The third shaft or outer jacket 628 surrounds the second shaft or outer sheath 624, and is translatably movable along it, such that a distal portion of the outer jacket 628 can be advanced to cover the blade 1012 of the cutting tip 632, as described below.

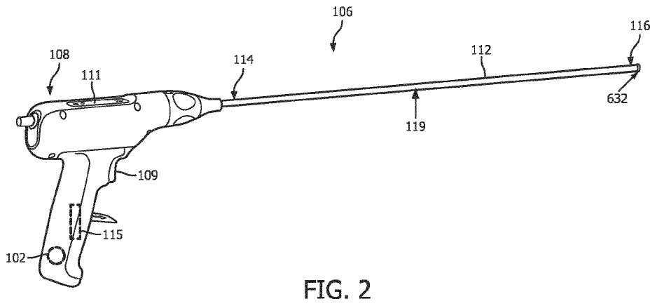

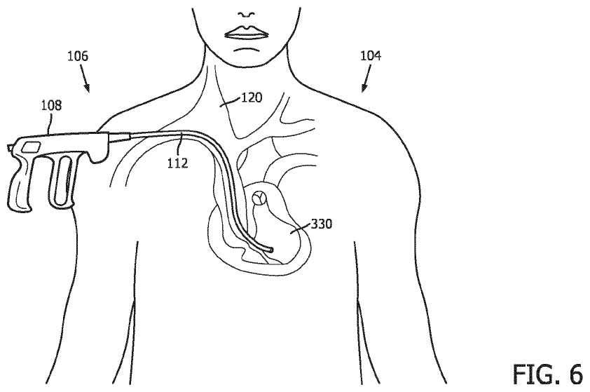

FIG. 2 is a side perspective view of an example surgical device 106, according to aspects of the present disclosure. The surgical device 106 includes a sheath assembly 112 that can be inserted into a body lumen 334 of a patient 104 (see FIG. 6). The sheath assembly includes a proximal portion 114 and a distal portion 118, separated by a working length 119 that is sufficient to perform the tasks described herein. The distal portion 118 includes a movable cutting tip 632.

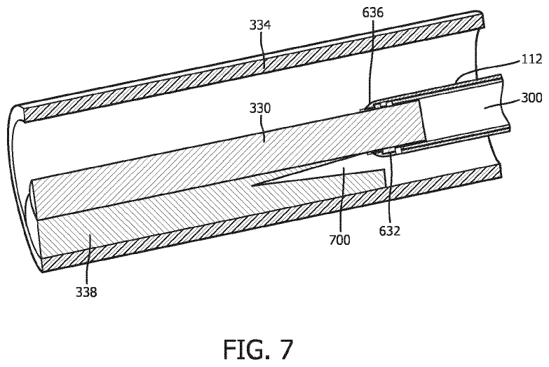

The sheath assembly 112 can surround an implanted lead 330 (see FIG. 7), such as a lead running along the left innominate vein past the superior vena cava (SVC) and connected into, or about, the right ventricle of the heart. Upon surrounding the lead 330 with the sheath assembly 112, the user of the surgical device 106 may actuate the handle assembly 108 (e.g., with a trigger 109), thereby actuating the cutting tip 632 located at the distal end of the sheath assembly 112, as described below. The actuated cutting tip 632, can then separate and/or cut the tissue surrounding an implanted lead within the body lumen of the patient.

Depending on the implementation, the handle assembly 108 may also include audiovisual (A/V) feedback indicators 111, a controller printed circuit board assembly (PCBA) 115, and a haptic feedback device 102.

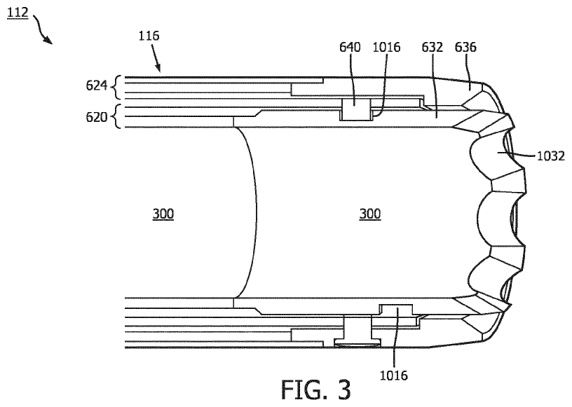

FIG. 3 is a side cross-sectional view of a distal portion 116 of an example sheath assembly 112, according to aspects of the present disclosure. The distal portion 116 of the sheath assembly 112 includes an outer band 636 fixedly attached to a flexible outer sheath 624, and a cutting tip 632 fixedly attached to a flexible inner sheath 620. In the example shown in FIG. 3, the flexible outer sheath 624 surrounds the flexible inner sheath 620, and the outer band 636 surrounds the cutting tip 632. The cutting tip 632 and flexible inner sheath 620 together define an inner lumen 300. A guide pin 640 is fixedly attached to the outer band 636. The cutting tip 632 is rotatably attached to the outer band 636 via the guide pin 640 that rests in a channel or can slot 1016. Activation of the trigger 109 of the handle assembly 108 (see FIG. 2) causes the flexible inner sheath 620 to rotate, whereas the flexible outer sheath 624 is rotationally fixed. The channel or cam slot 1016 is formed in the cutting tip 632 in a profile that varies in longitudinal distance with different radial positions, such that when the flexible inner sheath 620 is rotated while the flexible outer sheath 624 is rotationally fixed, the guide pin 640 travels through the cam slot 1016, causing the flexible inner shaft 620 and the cutting tip 632 to translate longitudinally as they rotate, as will be shown in greater detail below.

Depending on the profile of the cam slot 1016, a serrated blade or cutting surface 1012 of the cutting tip 632 may thus extend from and retract into the outer band 636 multiple times upon actuation of the trigger of the handle assembly. Depending on the implementation, the blade 1012 may rotate in a clockwise direction, a counterclockwise direction, or may oscillate between the two. When the clinician releases the trigger of the handle assembly, the blade 1012 of the cutting tip 632 may retract within the outer band 636, thereby allowing the clinician to force and advance the distal portion of the sheath assembly against additional uncut tissue, without engagement of the tissue by the blade 1012 of the cutting tip 632. The clinician may repeat the actuation step, thereby causing the blade 1012 of the cutting tip 632 to extend distally beyond the outer band 636 to cut the adjacent tissue. Each time actuation occurs, the proximal portion of the implanted lead and/or surrounding tissue enters further into the central lumen 300 of the sheath assembly 112. This process can be repeated until the surrounding tissue is completely or substantially dilated, and the implanted lead is separated and/or cut from the tissue. At that time, the implanted lead may safely be removed from the patient.

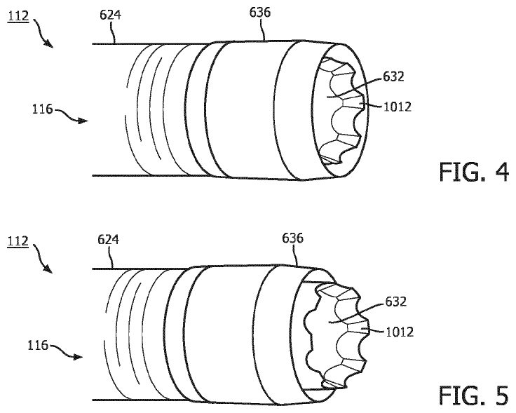

FIG. 4 is a perspective side view of the distal end of an example sheath assembly 112, according to aspects of the present disclosure. In this example, the blade 1012 of the cutting tip 632 is retracted inside the outer band 636. Also visible is the flexible outer sheath 624. In this configuration, the outer band 636 can be advanced against tissue in order to dilate or separate it without cutting (e.g., to separate the tissue from an implanted lead).

FIG. 5 is a perspective side view of the distal end of an example sheath assembly 112, according to aspects of the present disclosure. In this example, the blade 1012 of the cutting tip 632 is extended beyond the distal end of the outer band 636. Also visible is the flexible outer sheath 624. In this configuration, the blade 1012 can be advanced against tissue in order to cut it (e.g., to separate the tissue from an implanted lead).

FIG. 6 is a diagrammatic view of a surgical device 106 that has been introduced into a body lumen 334 of a patient 104 to remove an implanted lead 330, according to aspects of the present disclosure. The lead 330 may be surrounded by or embedded in tissue, which may be separated from the lead by the surgical device 106 as described herein. The lead may then be drawn into a lumen of the surgical device (e.g., lumen 300 of FIG. 3) for removal from the body lumen 334.

FIG. 7 is a side cross-sectional view of an example sheath assembly 112 removing a lead 330 from a body lumen 334, according to aspects of the present disclosure. After being implanted in the body lumen 334 for a period of time, the lead 330 may be partially or completely surrounded by tissue 338 (e.g., scar tissue) that has grown over the lead 330 within the body lumen 334. The tissue 338 may be attached or adhered to both the lead 330 and the wall of the body lumen 334, thus making the lead 330 difficult to safely remove from the body lumen 334.

In order to remove the lead safely, a clinician may advance the sheath assembly 112 may over a portion the lead 330 such that the lead 330 at least partially enters the lumen 300 of the sheath assembly 112. The sheath assembly may then be further advanced until it contacts the tissue 338, at which point the outer band 636 may be used to dilate the tissue, and/or the cutting tip 632 may be extended distal of the outer band such that the blade of the cutting tip (e.g., blade 1012 of FIG. 5) rotates and/or translates in contact with the tissue 338, thus cutting the tissue. Through a combination of dilation and cutting, the sheath assembly 112 may thus form a gap 700 between the tissue 338 and the lead 330. In cases where the tissue 338 completely surrounds the lead 330, the gap 700 may for example be a circular or cylindrical gap that is roughly concentric with the sheath assembly 112. When the gap 700 had been advanced past either an end of the lead 330 or an end of the tissue overgrowth 338, the lead may no longer be adhered, and may be safely removable from the body lumen 334.

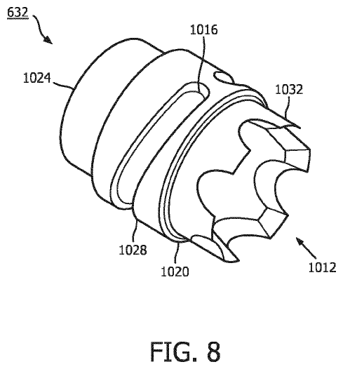

FIG. 8 is a side front perspective view of an example cutting tip 632, according to aspects of the present disclosure. The cutting tip 632 has a generally hollow cylindrical shape. The cutting tip 632 comprises a proximal portion 1024, an intermediate portion 1028, and a distal portion 1032. The outside diameter of the proximal portion 1024 is sized to allow it to be inserted to and/or engage (or otherwise attached to) the interior diameter of the inner flexible sheath (e.g., flexible inner sheath 620 of FIG. 3). The distal end of cutting tip 632 comprises a blade or cutting surface 1012, which may for example have a serrated, sharp blade profile. The intermediate portion 1028 comprises a channel or cam slot 1016 cut within its exterior surface.

As the inner flexible sheath rotates and translates within the outer sheath (e.g., flexible outer sheath 624 of FIG. 3), the outer sheath and pin may remain stationary. If so, the inner sheath, which is connected to cutting tip 632, forces the cutting tip 632 to rotate. The cam slot 1016 engages the guide pin, and the shape and profile of the cam slot 1016 controls the rate and distance with which the cutting tip 632 travels longitudinally. That is, the configuration of the cam slot 1016 controls the cutting tip's direction and amount of longitudinal travel as the cutting tip 632 is rotated, such as moving distally toward an extended position and/or proximally toward a retracted position, while the cutting tip 632 rotates in either a clockwise or counterclockwise direction.

In some aspects, the cutting tip 632 may also comprise a step up 1020 such that the outer diameter of the intermediate portion 1028 is greater than the outer diameter of the distal portion 1032, thus preventing the intermediate portion 1028 from fitting within the inner diameter of the inner sheath. As the cutting tip 632 rotates, and the blade or cutting surface 1012 extends beyond the distal end of the outer band into an extended position, the step up 1020 of the cutting tip 632 contacts the abutment of the outer band, thereby limiting the distance that the cutting tip 632 may travel and/or preventing the cutting tip 632 from exiting or extending beyond the distal tip of the outer sheath assembly, particularly the outer band, in the event that the guide pin is sheared.

The profile of the cam slot in the cutting tip may have various configurations, such as those disclosed in U.S. patent application Ser. No. 13/834,405 filed Mar. 15, 2013 and entitled Retractable Blade For Lead Removal Device, which is hereby incorporated herein by reference in its entirety as though fully set forth herein. For example, the cam slot 1016 may have a substantially linear profile, a substantially sinusoidal profile, or a combination of linear and non-linear profiles. Additionally, the cam slot 1016 may have an open and continuous configuration, thereby allowing the cutting tip to continuously rotate, or the cam slot may have a closed and discontinuous configuration such that when the cutting tip reaches its fully extended position, the trigger of the handle assembly may be released or reversed so that the cutting tip returns to initially retracted position before being re-actuated. For instance, the can slot 1016 in FIG. 8 is discontinuous because the cam slot does not travel around the entire circumference of the exterior of the cutting tip 632.

Although certain figures in this disclosure only illustrate either the open or closed cam slot configuration, either configuration may be used with any of the aspects disclosed and/or discussed herein and are considered within the scope of this disclosure. Furthermore, various types of cam slots 1016, such as a partial lobe cam (which includes a cam slot 1016 surrounding less than 360 degrees of the circumference of the exterior surface of the cutting tip 632), a single lobe cam (which includes a cam slot 1016 surrounding 360 degrees of the circumference of the exterior surface of the cutting tip 632), a double lobe cam (which includes a cam slot 1016 surrounding 720 degrees of the circumference of the exterior surface of the cutting tip 632) and/or other multiple lobe cams.

The distal end of cutting tip 632 may comprise a cutting surface 1012 having different blade profiles, such as those disclosed in U.S. patent application Ser. No. 13/834,405 filed Mar. 15, 2013 and entitled “Retractable Blade For Lead Removal Device”, which is hereby incorporated herein by reference in its entirety as though fully set forth herein. For example, the plane of the blade or cutting surface 1012 of the distal end 1032 of the cutting tip 632 depicted in the figures of this disclosure is parallel to the plane of the proximal end 1024 of the cutting tip 632. The plane of the cutting surface, however, may be offset (0 degrees to 90 degrees) from the plane of the proximal end 1024 of the cutting tip 623. Also, as discussed above, the profile of the cutting surface 1012 shown in FIG. 8 includes a plurality of serrations. However, depending on the implementation, the profile of the cutting surface 1012 need not be serrated, and may comprise other configurations, such as a constant and/or smooth sharp profile. The profile of the cutting surface 1012 in FIG. 8 includes 6 serrations. However, it may be desirable to have other numbers of serrations, such as 4, 5, 7, 8, 10, or more serrations. Furthermore, the serrations may comprise a myriad of different shapes and configurations, including but not limited to any variation of a square, rectangle, rhombus, parallelogram, trapezoid, triangle, circle, ellipse, kite, etc.

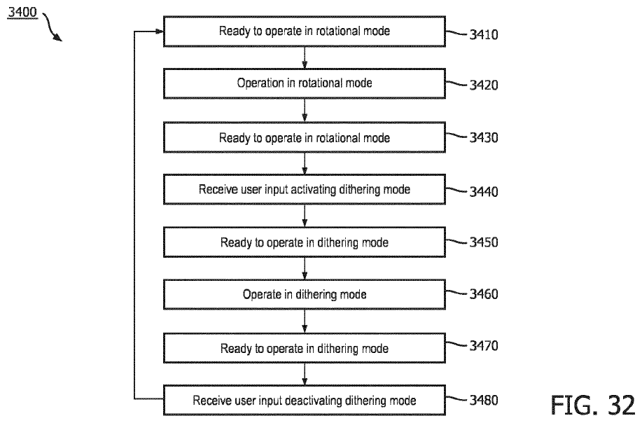

As discussed above, FIG. 8 depicts the intermediate portion 1028 of the cutting tip 632 having a cam slot (or channel) 1016 cut within its exterior surface, as a means of controlling longitudinal motion of the cutting tip 632 as the cutting tip 632 is rotated. It should be understood that this mechanism is presented here for exemplary purposes, and that other means of rotating and/or translating the cutting tip 632 may be used instead or in addition, without departing from the spirit of the present disclosure, so long as at least some of the methods described herein (e.g., in any of FIGS. 32-37) can be performed.

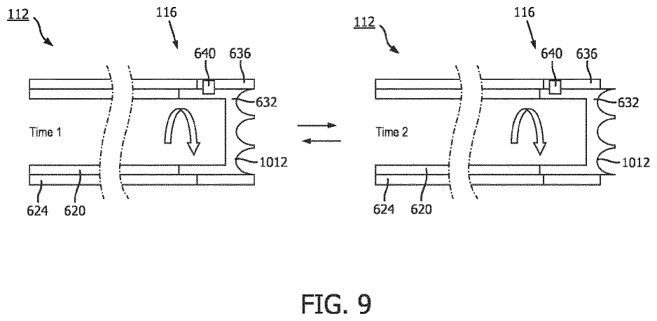

FIG. 9 is a schematic cross-sectional view of the reciprocating motion of a cutting tip 632 of the distal portion 116 of an example sheath assembly 112, according to aspects of the present disclosure. Visible is the guide pin 640, which is fixedly attached to the outer band 636, which is fixedly attached to a distal end of the flexible outer sheath 624. The flexible outer sheath 624 surrounds the flexible inner sheath 620, to whose distal end the cutting tip 632 is fixedly attached. Due to the motion of the guide pin 640 in the cam slot 1016 as the flexible inner sheath and cutting tip 632 (as described above), the cutting tip 632 may move longitudinally as it rotates, such that at a first time (“Time 1”) the blade 1012 of the cutting tip 632 is in a retracted position within the outer band 636, while at a second time the blade 1012 of the cutting tip 632 is in an extended or cutting position wherein the blade 1012 projects beyond a distal end of the outer band 636. Depending on the implementation, rotation of the cutting tip 632 relative to the outer band 636 may cause the blade 1012 to oscillate between the extended and retracted positions, either by continuous rotation in one direction (e.g., clockwise or counterclockwise) or by oscillating rotation in alternating directions. In some examples, the retracted position may represent a “home” position for the blade 1012, such that when the trigger of the handle assembly is released, the flexible inner sheath 620 and the cutting tip 632 are automatically rotated to a “home” clock angle wherein the cutting tip 632 is translated to a longitudinal position wherein the blade 1012 of the cutting tip 632 is behind the outer band 636 and thus protected from cutting tissues of the patient.

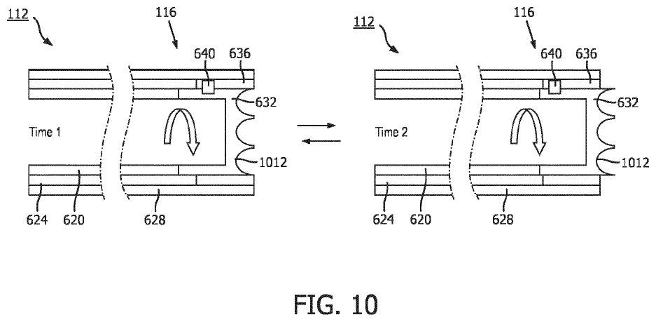

FIG. 10 is a schematic cross-sectional view of the reciprocating motion of a cutting tip 632 of the distal portion 116 of the example sheath assembly 112 of FIG. 9, according to aspects of the present disclosure. Visible are the guide pin 640, outer band 636, flexible outer sheath 624, flexible inner sheath 620, cutting tip 632, and blade 1012. Also visible in FIG. 10 is an outer jacket or guard 628, which surrounds the flexible outer sheath 624. The outer jacket or guard 628 may extend from a proximal end to a distal end of the sheath assembly 112, and may be manually extendable or retractable by the clinician, such that in its fully retracted position (shown here in FIG. 10) the outer jacket or guard 628 does not extend beyond the distal end of the outer band 636. In a fully extended position (shown below in FIG. 11), a distal end of the outer jacket or guard 628 may extend distal of the distal end of the outer band 636. In this configuration, the reciprocating action of the cutting tip 623 and blade 1012 can proceed as described above in FIG. 9.

FIG. 11 is a schematic cross-sectional view of the reciprocating motion of a cutting tip 632 of the distal portion 116 of the example sheath assembly 112 of FIG. 10, according to aspects of the present disclosure. Visible are the guide pin 640, outer band 636, flexible outer sheath 624, flexible inner sheath 620, cutting tip 632, and blade 1012. Also visible in FIG. 11 is the outer jacket or guard 628, which surrounds the flexible outer sheath 624. In the example of FIG. 11, the outer jacket or guard 628 is in an extended position, such that the distal end of the outer jacket or guard 628 extends distal of the distal end of the outer band 636, by an amount sufficient to cover the blade 1012 of the cutting tip 632, even when the blade 1012 is in its fully extended position. A clinician may for example place the outer jacket or guard 628 in this position such that if an accidental trigger press occurs, resulting in rotation and longitudinal translation of the cutting tip 632, the blade 1012 will nevertheless be protected from cutting tissues of the patient. In other instances, the shield may permit the blade to cut tissue while shielded, reducing potential to cut the vessel wall or lead. Tissue may be pulled into the cutting mechanism in this manner.

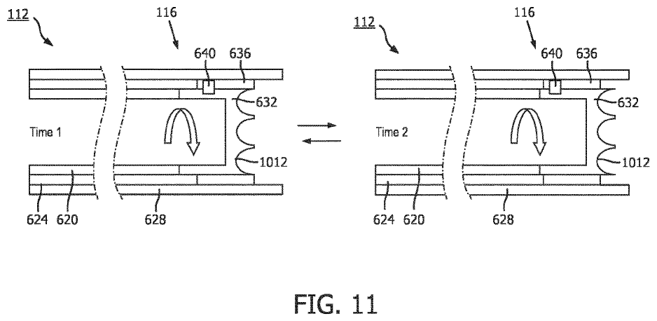

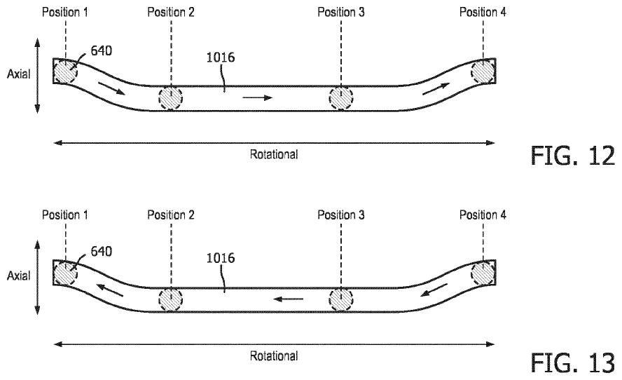

FIG. 12 is a schematic representation of a guide pin 640 moving rightward (e.g., clockwise as seen looking proximally from a distal end of the cutting tip 632) in an example cam channel 1016, according to aspects of the present disclosure. It should be understood that a rotation and longitudinal translation of the cutting tip 632 and cam channel or cam slot 1016 with respect to the guide pin 640, while the guide pin 640 remains fixed, can also be conceived, modeled, or described as a movement of the guide pin 640 within the cam channel 1016. For convenience, these descriptions will be used interchangeably, even where it is understood that the guide pin 640 may not actually be moving. The motor is coupled to the flexible inner shaft via an actuator or power train assembly (e.g., one or more gears). The motor rotates gears, which rotate the flexible inner shaft. Rotation of inner shaft causes rotation of the cutting tip 632 and cam slot 1016, which causes movement of the cam pin or guide pin 640 within the cam slot.

The example of FIG. 12 shows a first mode or rotational mode, wherein the configuration of the cam slot (whether continuous or discontinuous around the circumference of the cutting tip 632) provides combined axial and rotational movement along the entire path of the cam slot 1016. Position 2 and position 3 of the guide pin 640 both occur in a straight, flat portion of the cam slot 1016, such that a movement of the guide pin between position 1 and position 2 (e.g., caused by rotation of the cutting tip 632 relative to the outer band 636) will not result in axial motion of the cutting tip 632 relative to the outer band 636. It is noted that axial movement can also be reference to as longitudinal movement (e.g., axial movement along longitudinal axis), linear movement, translational movement, etc.

Positions 1 and 4 are found at different axial positions than Positions 2 and 3, such that movement of the guide pin 640 from position 1 to position 2 or from position 3 to position 4 does result in an axial movement of the cutting tip 632. However, for most of the path of the cam slot (e.g., most of the rotation of the cutting tip 632), there will be no axial movement, and thus the particular configuration of the cam slot 1016 shown in FIG. 12 may be considered a primarily rotational configuration, with some limited axial motion occurring in certain regions. Depending on the needs of the clinician and the exact configuration of the lead and the tissue overgrowth within the patient's body lumen, such a configuration may be desirable. However, in other circumstances the clinician may wish to emphasize axial motion over rotational motion. Such modes will be described below.

FIG. 13 is a schematic representation of a guide pin 640 moving leftward (e.g., counterclockwise as seen looking proximally from a distal end of the cutting tip) in the example cam channel 1016 of FIG. 12, according to aspects of the present disclosure. In many operational modes, the cam channel 1016 is discontinuous around the cutting tip, and drive motors in the handle section of the surgical instrument may oscillate back and forth between clockwise and counterclockwise rotation of the flexible inner sheath 620, and thus oscillating between leftward and rightward movement of the guide pin 640 in the cam slot 1016. As described above, the cam slot 1016 shown in FIGS. 12 and 13 is configured primarily for rotational motion, with some axial motion near the ends of its travel. Thus, oscillating between clockwise and counterclockwise rotation will result in an oscillating axial motion of the cutting tip blade. In other examples, the cam channel 16 may be continuous around the circumference of the cutting tip, and an oscillating axial motion of the cutting tip blade can be achieved by continuous rotation in a single direction.

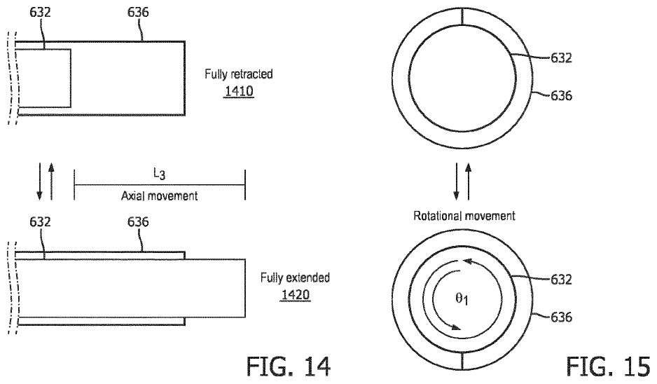

FIG. 14 is a schematic side cross-sectional view of the reciprocating motion of a cutting tip 632, according to aspects of the present disclosure. In the first mode or rotational mode described above, the path of the cam slot provides for combined axial and rotational movement along entire cam path, but emphasizes rotational movement. Nevertheless, as described above, rotation of the cutting tip 632 relative to the outer band 636 results in an oscillating axial motion of the cutting tip 632 between a fully retracted position 1410 and a fully extended position 1420, over a travel length L₁. Depending on the implementation, the cutting tip 632 may or may not be shielded by a guard or outer jacket 628 (see FIG. 11) during this axial oscillation. It is noted that combined rotation and translation of the blade 1012 causes front and side sharp edges of the blade 1012 to slice tissue, whereas longitudinal translation of blade causes front sharp edges of the blade1012 to impact tissue.

FIG. 15 is a schematic front cross-sectional view of the reciprocating motion of a cutting tip 632, according to aspects of the present disclosure. In the first mode or rotational mode, the axial oscillation of the cutting tip 632 relative to the outer band 636, as described in FIG. 14, is caused by an oscillating rotational movement (e.g., alternating between clockwise and counterclockwise rotation) of the cutting tip 632 relative to the outer band 636, through a travel angle θ₁.

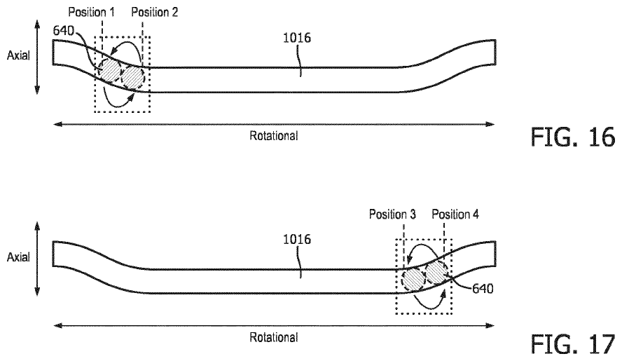

FIG. 16 is a schematic representation of a guide pin 640 oscillating leftward and rightward in an example cam channel 1016, according to aspects of the present disclosure. In this configuration, a first position, Position 1, and a second position, and Position 2, are located on a portion of the cam path that exposes the blade of the cutting tip, and are also located at a portion of the cam path 1016 that has the steepest slope. The result is a second mode or impact mode, which yields combined axial and rotational movement along a small segment of cam path, and emphasizes axial movement to the maximum extent permitted by the given shape of the cam slot 1016. In the configuration shown, the slope of the cam slot 1016 between Position 1 and Position 2 is approximately 45 degrees, resulting in a balance between rotational and axial motion. It is noted that for cam paths 1016 with steeper slopes (e.g., slopes greater than 45 degrees), greater axial motion of the blade can be generated by smaller rotational motion of the cutting tip.

FIG. 17 is a schematic representation of a guide pin 640 oscillating leftward and rightward in an example cam channel 1016, according to aspects of the present disclosure. In this configuration, the first position, Position 3, and second position, and Position 4, are located on a portion of the cam path that is opposite to, but symmetrical with, Positions 1 and 2 shown in FIG. 16. In an example, the cam paths may not be 180 degrees apart, but may be symmetrically opposite to the cam path centerline, for both continuous and discontinuous cam paths. However, it is understood that the conditions of symmetry and continuity are not required to effectuate the reciprocating motions described herein. In the example shown in FIGS. 16 and 17, the cam slot 1016 is left-right symmetrical, and thus oscillation between Positions 3 and 4 will produce an axial motion similar to (though out of phase with) the axial motion of FIG. 17. In some aspects, an impact mode may involve oscillation in two separate regions of the cam path 1016, such as those shown in FIGS. 16 and 17. For example, oscillation in a first region for a first period of time may then be followed by oscillation in a second (e.g., perhaps symmetric) region for a second period of time.

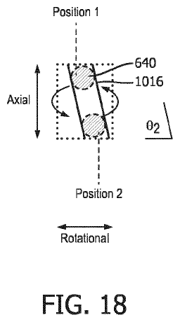

FIG. 18 is a schematic representation of a guide pin 640 oscillating upward and downward in an example cam channel 1016, according to aspects of the present disclosure. In this configuration, the first position, Position 1, and the second position, are in a portion of the cam channel that is angled steeply, resulting in a large axial motion of the blade for a small or rotational motion of the cutting tip. Thus, the second mode or impact mode is enhanced in this configuration (e.g., there is much more axial motion than rotational motion). In some instances, the cam channel is curved. For example, the cam pathway can have a gentle or gradual, non-linear change in slope. At any given point along the cam channel, the slope can be between 0 degrees to 89 degrees. A nearly vertical cam channel may for example have a slope between 45 and 89 degrees, plus or minus five degrees. A gently sloped cam channel may for example have a slope between 0 degrees and 45 degrees, plus or minus five degrees. In other instances, a nearly vertical cam channel may have a slope between 60 degrees and 80 degrees. It is understood that similar path shapes may or may not be found at an opposite side the of cam slot 1016 (whether symmetrically or otherwise).

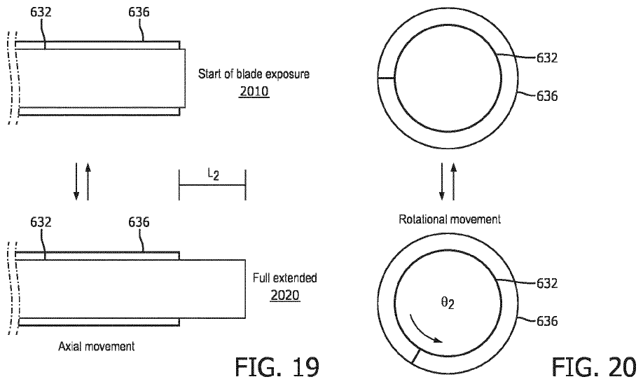

FIG. 19 is a schematic side cross-sectional view of the reciprocating motion of a cutting tip 632, according to aspects of the present disclosure. In the second mode or impact mode there is combined axial and rotational movement along segment of cam path, but with particular emphasis on axial movement, such that rotation of the cutting tip 632 relative to the outer band 636 results in an oscillating axial motion of the cutting tip 632 between an exposed blade position 2010 and a fully extended blade position 2020, over a travel length L2 that is less that the length L1 shown in FIG. 14. Thus, the impact mode oscillation shown here in FIG. 20 may occur at a higher frequency than the rotational mode oscillation shown in FIG. 14. In some instances, a higher motor speed may also be used in impact mode in order to further increase the oscillation frequency, which may improve the efficacy of the impact mode in cutting tissue. Frequency and/or oscillation frequency can also be referenced as cycle frequency.

Depending on the implementation, the cutting tip 632 may or may not be shielded by a guard or outer jacket 628 (see FIG. 11) during this axial oscillation.

In an example, cam path geometry for a commercially available cutting tip retracts or exposes the cutting tip up to 0.037 inches within ~70 degree rotation, with blade exposure beginning after 0.024 inches of axial extension, at ~45 degrees of rotation. It is therefore possible for the cam cutter to move 0.11 inches axially with only 25 degrees of rotation. Using a type of processor called a programmable motor controller, it is possible to quickly alternate back and forth within this region of the cam path, resulting in a recurrent axial motion. Furthermore, as there are two regions where this may occur (region 1a from 45°-70°, and region 1b from 190°-315°). By alternating performance of the movement between the first region and an equivalent second region, the designer can reduce fatigue to the cam pin/path. Example pseudo code for this implementation is:

```
While (trigger_is_pulled, and axial_mvmt_flag_is_set):
  if(region_flag == region_1a):
    f_Alternate_movement(45,70)
      region_flag = region_1b
  elseif(region_flag == region_1b):
    f_Alternate_movement(290,315)
    region_flag = region_1a
  ENDIF
END_while
function f_alternate_movement(from_location_in_deg,
to_location_in_deg):
store_location_variable = to_location_in_deg
  if(motor_is_ready && trigger_is_pulled):
  motor.move(to_location_in_deg)
to_location_in_deg = from_location_in_deg
from_location_in_deg = store_location_variable
  if(trigger_is_pulled == FALSE):
    break function
  else
    f_alternate_movement(from_location_in_deg,
    to_location_in_deg)
  ENDIF
END_FUNCTION
```

The while loop of the function ensures that the dilation trigger is pulled (user input in provided), and that axial movement is intended (may be set via double-tapping the trigger in extended mode, or other means of selection); if this is the case, the device identifies which region to perform axial movement, and runs the associated function. Upon completing the function, the alternate region flag is selected to reduce pin wear. The function, as written, uses recursion to alternate movement with minimal delay. Once the trigger is released, the function breaks (e.g., exits), and returns to the while loop. The motor.move( ) may provide motor movement via the shortest angular path to the specified location.

It is noted that many way may exist to implement this code, and they may be functionally equivalent to or even functionally indiscernible from one another. Generally, recursion may be the fastest code implementation for embedded systems, and may reduce the amount of memory (ROM and RAM) required on board the surgical device. The use of While loops may not be advisable under some conditions, but for purposes of this disclosure, the While loop may be considered a reasonable proxy actual implementation (via state machine handling, finite for loops, interrupt handling, or other logical construction to enforce device state). It is further noted that equivalent functionality may be implemented using hardware only, and thus the pseudo code disclosed herein serves to describe the functionality rather than to limit its implementation.

This aspect may be generalized to any particular cam path. The cam path may be continually traversable (the cam path loops back on itself). The cam path is not required to be continually traversable (i.e., it is not required to loop back on itself) for this methodology to work. Use of the impact mode may not be constrained to intracardiac device (ICD) lead removal, but can find usefulness in other areas such as intraluminal, intravascular, and intracardiac, excision, biopsy, Targeted Lead Extraction (TLE), or other applications where a cutting catheter may be used.

FIG. 20 is a schematic front cross-sectional view of the reciprocating motion of a cutting tip 632, according to aspects of the present disclosure. In the second mode or impact mode, the axial oscillation of the cutting tip 632 relative to the outer band 636, as described in FIG. 20, is caused by an oscillating rotational movement (e.g., alternating between clockwise and counterclockwise rotation) of the cutting tip 632 relative to the outer band 636, through a travel angle θ₂, which is less than the angle θ₁ of FIG. 15. In some aspects, as shown for example in FIG. 18, the oscillating motion of FIG. 20 may be achieved with little or no rotation (e.g., θ₂≈0).

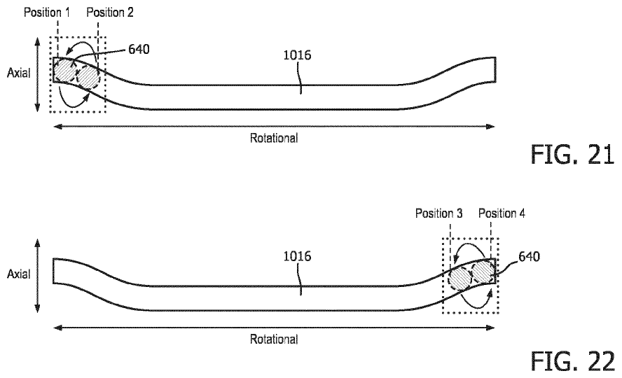

FIG. 21 is a schematic representation of a guide pin 640 oscillating leftward and rightward in an example cam channel 1016, according to aspects of the present disclosure. In this configuration, a first position, Position 1, and a second position, and Position 2, are located on a portion of the cam path that maximizes vibration (whether axial or torsional) of the cutting tip. The result is a second mode, mobility mode, or dithering mode, which yields combined axial and rotational movement along a small segment of cam path, and emphasizes vibration of the cutting blade, outer band, and flexible inner sheath. Such vibration may be useful for example in loosening the implanted lead from tissue overgrowth, or in encouraging the motion of the lead and/or cut tissue through the lumen of the flexible inner sheath toward the proximal end of the sheath assembly. In some cases, Position 1 and Position 2 may be selected to be closer together than in (for example) the second mode or impact mode, in order to generate a higher oscillation frequency. In other cases, instead or in addition, the dithering mode may be run at a higher motor speed in order to increase the oscillation frequency.

Depending on the implementation, the motion of the dithering mode may include rotational motion only (shallow or zero slope), axial movement only (very high slope), or combined axial and rotational movement (intermediate slope), in order to maximize a desired vibration mode. Depending on the implementation, Position 1 and Position 2 may be positions along the cam slot 1016 that do or do not expose the blade of the cutting tip, or a combination thereof. The dithering mode may or may not be implemented with a guard or outer jacket 628 shielding the blade 1012 (see FIG. 11). In some instances, the guard or outer jacket 628 may completely shield the blade when the blade is at its full dither-mode retraction, and may completely shield the blade when the blade is at its full dither-mode extension (as shown in, e.g., FIG. 11). In such instances, operating the cutting sheath in the dithering mode is completely dedicated to facilitating movement of the lead or other medical device (e.g., guidewire, catheter, guide catheter, etc.) relative to the flexible elongate member of the cutting sheath. In particular, dithering mode allows oscillation of the inner sheath 620 (FIG. 1) and/or the cutting tip 632 relative to the lead or other medical device so that the medical device does not get stuck inside the lumen defined by the inner sheath 620 and instead has smooth, consistent longitudinal movement relative to the cutting sheath. Because the cutting tip 632 is shielded completely during this oscillation, the dithering mode is unrelated to cutting tissue. In other instance, dithering mode can also be associated with cutting tissue, e.g., because the blade is partially or completely exposed (as well as facilitating longitudinal movement of the lead or other medical device relative to the cutting sheath).

FIG. 22 is a schematic representation of a guide pin 640 oscillating leftward and rightward in an example cam channel 1016, according to aspects of the present disclosure. In this configuration, Positions 3 and 4 are the mirror image of Positions 1 and 2 in FIG. 22. It is understood that the cam slot or cam path 1016 may not be symmetrical, and thus positions on one side of the cam slot 1016 may not produce equivalent vibrational motion to equidistant positions on the opposite side of the cam slot 1016.

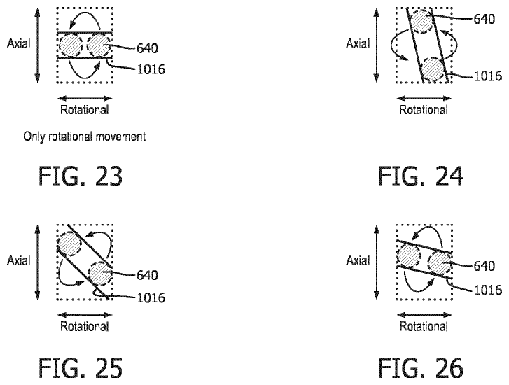

In some aspects, a dithering mode may involve oscillation in two separate regions of the cam path 1016, such as those shown in FIGS. 22 and 23. For example, oscillation in one region for a period of time may then be followed by oscillation in a different (e.g., perhaps symmetric) region for a second period of time.

FIG. 23, FIG. 24, FIG. 25, and FIG. 26 show different slopes of the cam path 1016 that may be selected for the dithering mode, according to aspects of the present disclosure. As described above, shallower slopes (e.g., slopes between 0 and 45 degrees) are associated with greater rotational motion and less axial motion, whereas steeper slopes (e.g., slopes between 45 and 89 degrees) are associated with greater axial motion and less rotational motion. In some aspects, the stop positions, Position 1 and Position 2 on the cam slot 1016, may be hard-wired or hard-coded into the surgical instrument. In other aspects, a selection of different vibration modes may be offered to a user, each controlled by different stop positions along the cam slot 1016. In still other aspects, Positions 1 and 2 may be user-selectable in order to customize a desired vibration mode for the surgical instrument. It is understood that similar or mirror-image stop positions or cam paths may be used instead or in addition to those shown here.

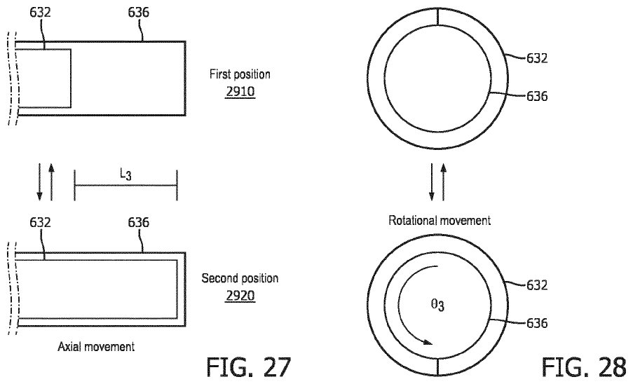

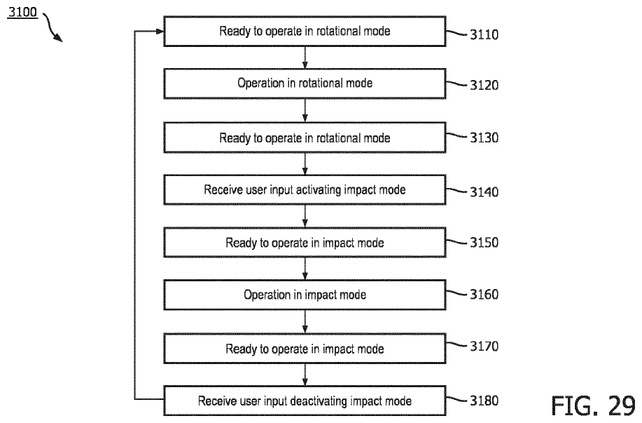

FIG. 27 is a schematic side cross-sectional view of the reciprocating motion of a cutting tip 632, according to aspects of the present disclosure. In the third mode or dithering mode, there may be axial and/or rotational movement along segment of cam path, but with particular emphasis on desirable vibration modes, such that rotation of the cutting tip 632 relative to the outer band 636 results in an oscillating axial motion of the cutting tip 632 between first blade position 2910 and a second blade position 2020, over a travel length L3 that may be greater than, equal to, or less than the length L2 shown in FIG. 20. The dither-mode oscillation shown here in FIG. 29 may occur at a higher frequency than the rotational mode oscillation shown in FIG. 14, and may also occur at a higher frequency than the impact mode oscillation shown in FIG. 20. In some instances, a higher motor speed may also be used in dithering mode in order to further increase the oscillation frequency, which may improve the efficacy of the dithering mode in moving leads and/or tissue through the lumen of the flexible inner sheath 620.

In some examples, neither the first position nor the second position exposes the blade of the cutting tip 632. In other examples, both the first position and the second position expose the blade of the cutting tip 632. In still other examples, the first position may not expose the blade, while the second position does expose the blade. Depending on the implementation, the cutting tip 632 may or may not be shielded by a guard or outer jacket 628 (see FIG. 11) during dithering movement.

Use of the dithering mode may not be constrained to intracardiac device (ICD) lead removal, but can find usefulness in other areas such as intraluminal, intravascular, and intracardiac, excision, biopsy, Targeted Lead Extraction (TLE), or other applications where a cutting catheter may be used. In some aspects, movement of the flexible elongate member (e.g., the cutting catheter) itself may have clinically significant implications for capturing the tissue attached to a lead.

FIG. 28 is a schematic front cross-sectional view of the reciprocating motion of a cutting tip 632, according to aspects of the present disclosure. In the third mode or dithering mode, the axial oscillation of the cutting tip 632 relative to the outer band 636, as described in FIG. 29, is caused by an oscillating rotational movement (e.g., alternating between clockwise and counterclockwise rotation) of the cutting tip 632 relative to the outer band 636, through a travel angle θ₃, which is less than the angle θ₁ of FIG. 15, but may be greater than, equal to, or less than the angle θ₂ of FIG. 21. In some aspects, as shown for example in FIG. 24, the oscillating motion of FIG. 29 may be achieved with little or no rotation (e.g., θ₃≈0).

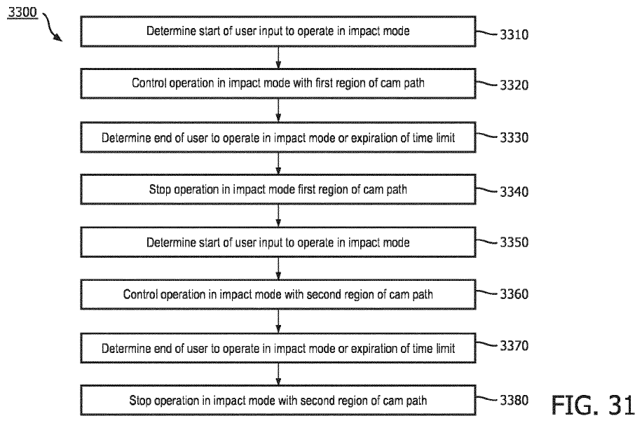

FIG. 29 illustrates a flow diagram for an example method 3100 for transition from a rotational mode to an impact mode, according to aspects of the present disclosure. It is understood that the steps of method 3100 may be performed in a different order than shown in FIG. 31, additional steps can be provided before, during, and after the steps, and/or some of the steps described can be replaced or eliminated in other aspects. These steps may be executed in real time or near-real time, for example as coded instructions on a processor such as processor circuit 3850 of FIG. 38, in response to inputs by a clinician or other user.

In step 3110, the surgical device is ready to operate in rotational mode.

In step 3120, in response to a first user input (e.g., a trigger press to a first trigger position), the method includes beginning to operate the surgical device in rotational mode as described above.

In step 3130 the method includes, while the surgical instrument is operating in rotational mode, awaiting a user input to either continue operating in the rotational mode or switch the surgical device to an impact mode.

In step 3140, the method includes receiving a second user input (e.g., a trigger press to a second trigger position) to switch the surgical device to the impact mode.

In step 3150, the method includes configuring the surgical device for operation in the impact mode (e.g., by rotating the cutting tip to a particular position).

In step 3160, the method includes operating the surgical device in the impact mode as described above.

In step 3170 the method includes, while the surgical instrument is operating in impact mode, awaiting a user input to either continue operating in the impact mode or deactivate the impact mode and switch the surgical device back to the rotational mode.

In step 3180 the method includes receiving a third user input (e.g., a trigger release from the second trigger position to the first trigger position) to exit or deactivate the impact mode. Execution then returns to step 3110.

A person of ordinary skill in the art will understand that for some aspects, one or more of the above steps could be eliminated, combined, or performed in a different sequence, and that other steps may be added.

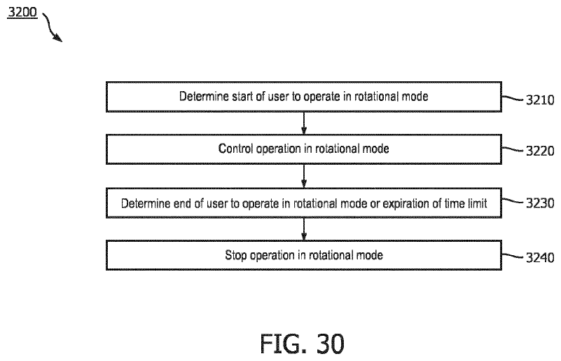

FIG. 30 illustrates a flow diagram for an example method 3200 for operation in a rotational mode, according to aspects of the present disclosure. It is understood that the steps of method 3200 may be performed in a different order than shown in FIG. 32, additional steps can be provided before, during, and after the steps, and/or some of the steps described can be replaced or eliminated in other aspects. These steps may be executed in real time or near-real time, for example as coded instructions on a processor such as processor circuit 3850 of FIG. 38, in response to inputs by a clinician or other user.

In step 3210, the method includes receiving a user input (e.g., a trigger press) to start operation in the rotational mode.

In step 3220, the method includes operating the surgical device in the rotational mode until the conditions of step 3230 are met.

In step 3230, the method includes either receiving a user input (e.g., a trigger release) to end operation in the rotational mode, or exceedance/expiration of a time limit since the start of the rotational mode. Such a time limit may, for example, reduce the chance of overheating the drive motors of the surgical device.

In step 3249, the method includes ceasing operation of the surgical device in the rotational mode.

A person of ordinary skill in the art will understand that for some aspects, one or more of the above steps could be eliminated, combined, or performed in a different sequence, and that other steps may be added.

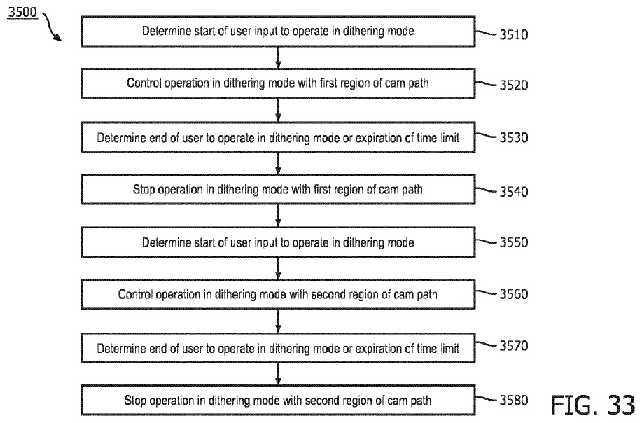

FIG. 31 illustrates a flow diagram for an example method 3300 operation in an impact mode, according to aspects of the present disclosure. It is understood that the steps of method 3300 may be performed in a different order than shown in FIG. 33, additional steps can be provided before, during, and after the steps, and/or some of the steps described can be replaced or eliminated in other aspects. These steps may be executed in real time or near-real time, for example as coded instructions on a processor such as processor circuit 3850 of FIG. 38, in response to inputs by a clinician or other user.

In step 3310, the method includes receiving a user input (e.g., a trigger press) to begin operation in the impact mode.

In step 3320, the method includes operating the surgical device in the impact mode by keeping the guide pin in a first region of the cam path.

In step 3330, the method includes either receiving a user input (e.g., a trigger release) to end operation in the impact mode, or exceedance/expiration of a time limit since the start of the impact mode. Such a time limit may, for example, reduce the chance of overheating the drive motors of the surgical device.

In step 3340, the method includes ceasing to operate the surgical device in the impact mode.

In step 3350, the method includes again receiving the user input to operate the surgical device in the impact mode.

In step 3360, the method includes operating the surgical device in the impact mode by keeping the guide pin in a second region of the cam path. By alternating between two different regions of the cam path, the method may for example reduce wear on components of the surgical device.

In step 3370, the method includes again either receiving the user input to end operation in the impact mode, or exceedance/expiration of the time limit since the start of the impact mode.

In step 3380, the method includes ceasing to operate the surgical device in the impact mode.

A person of ordinary skill in the art will understand that for some aspects, one or more of the above steps could be eliminated, combined, or performed in a different sequence, and that other steps may be added.

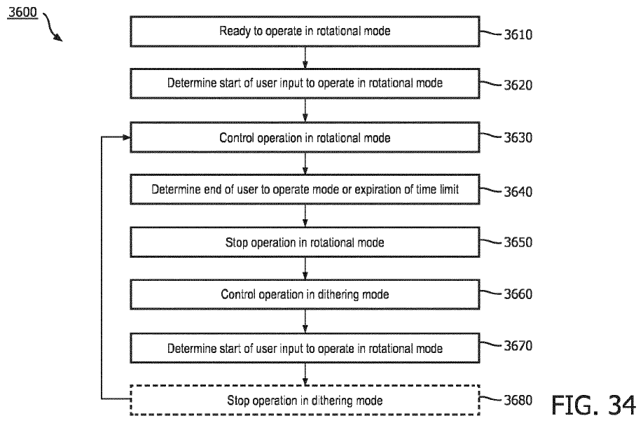

FIG. 32 illustrates a flow diagram for an example method 3400 for transition from a rotational mode to a mobility or dithering mode, according to aspects of the present disclosure. It is understood that the steps of method 3400 may be performed in a different order than shown in FIG. 34, additional steps can be provided before, during, and after the steps, and/or some of the steps described can be replaced or eliminated in other aspects. These steps may be executed in real time or near-real time, for example as coded instructions on a processor such as processor circuit 3850 of FIG. 38, in response to inputs by a clinician or other user.

In step 3410, the surgical device is ready to operate in rotational mode.

In step 3420, in response to a first user input (e.g., a trigger press to a first trigger position), the method includes beginning to operate the surgical device in rotational mode as described above.

In step 3430 the method includes, while the surgical instrument is operating in rotational mode, awaiting a user input to either continue operating in the rotational mode or switch the surgical device to an impact mode.

In step 3440, the method includes receiving a fourth user input (e.g., a trigger press to a third trigger position) to switch the surgical device to the mobility or dithering mode.

In step 3450, the method includes configuring the surgical device for operation in the mobility or dithering mode (e.g., by rotating the cutting tip to a particular position).

In step 3460, the method includes operating the surgical device in the mobility or dithering mode as described above.

In step 3470 the method includes, while the surgical instrument is operating in mobility or dithering mode, awaiting a user input to either continue operating in the mobility/dithering mode or deactivate the mobility/dithering mode and switch the surgical device back to the rotational mode.

In step 3480 the method includes receiving a fifth user input (e.g., a trigger release from the third trigger position to the first trigger position) to exit or deactivate the impact mode. Execution then returns to step 3110.

A person of ordinary skill in the art will understand that for some aspects, one or more of the above steps could be eliminated, combined, or performed in a different sequence, and that other steps may be added.

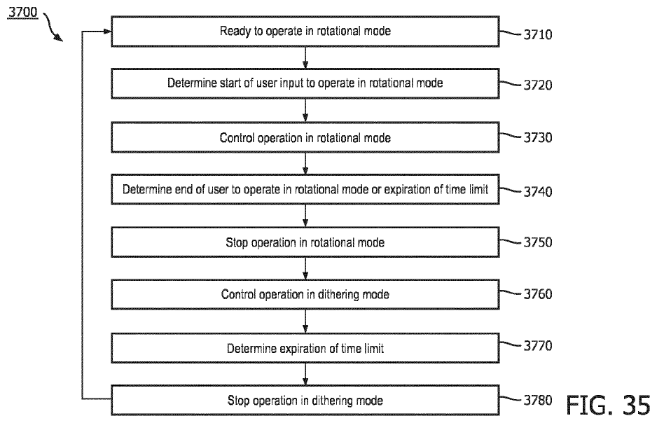

FIG. 33 illustrates a flow diagram for an example method 3500 for operation in a mobility or dithering mode, with time limit, according to aspects of the present disclosure. It is understood that the steps of method 3500 may be performed in a different order than shown in FIG. 35, additional steps can be provided before, during, and after the steps, and/or some of the steps described can be replaced or eliminated in other aspects. These steps may be executed in real time or near-real time, for example as coded instructions on a processor such as processor circuit 3850 of FIG. 38, in response to inputs by a clinician or other user.

In step 3510, the method includes receiving a user input (e.g., a trigger press) to begin operation in the mobility or dithering mode.

In step 3520, the method includes operating the surgical device in the mobility or dithering mode by keeping the guide pin in a first region of the cam path.

In step 3530, the method includes either receiving a user input (e.g., a trigger release) to end operation in the mobility or dithering mode, or exceedance/expiration of a time limit since the start of the mobility or dithering mode. Such a time limit may, for example, reduce the chance of overheating the drive motors of the surgical device.

In step 3540, the method includes ceasing to operate the surgical device in the mobility or dithering mode.

In step 3550, the method includes again receiving the user input to operate the surgical device in the mobility or dithering mode.

In step 3560, the method includes operating the surgical device in the mobility or dithering mode by keeping the guide pin in a second region of the cam path. By alternating between two different regions of the cam path, the method may for example reduce wear on components of the surgical device.

In step 3570, the method includes again either receiving the user input to end operation in the mobility or dithering mode, or exceedance/expiration of the time limit since the start of the mobility or dithering mode.

In step 3580, the method includes ceasing to operate the surgical device in the mobility or dithering mode.

A person of ordinary skill in the art will understand that for some aspects, one or more of the above steps could be eliminated, combined, or performed in a different sequence, and that other steps may be added.

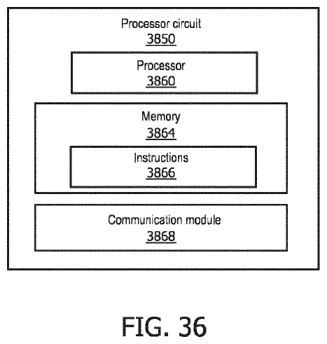

FIG. 34 illustrates a flow diagram for an example method 3600 for switching between a rotational mode and a default mobility or dithering mode, with time limits, according to aspects of the present disclosure. It is understood that the steps of method 3600 may be performed in a different order than shown in FIG. 36, additional steps can be provided before, during, and after the steps, and/or some of the steps described can be replaced or eliminated in other aspects. These steps may be executed in real time or near-real time, for example as coded instructions on a processor such as processor circuit 3850 of FIG. 38, in response to inputs by a clinician or other user.

In step 3610, the method includes waiting for a user input to begin operating the surgical device in the rotational mode.

In step 3620, the method includes receiving a user input (e.g., a trigger press) to begin operation in the rotational mode.

In step 3630, the method includes operating the surgical device in the rotational mode.

In step 3640, the method includes either receiving a user input (e.g., a trigger release) to end operation in the rotational, or exceedance/expiration of a time limit since the start of the rotational mode. Such a time limit may, for example, reduce the chance of overheating the drive motors of the surgical device.

In step 3650, the method includes ceasing to operate the surgical device in the rotational mode.

In step 3660, the method includes operating the device in the default mobility or dithering mode.

In step 3670, the method includes either receiving a user input (e.g., a trigger release) to end operation in the mobility or dithering mode, or exceedance/expiration of a time limit since the start of the mobility or dithering mode. Such a time limit may, for example, reduce the chance of overheating the drive motors of the surgical device.

In step 3680, the method includes ceasing to operate the surgical device in the mobility or dithering mode. Execution then returns to step 3630.

A person of ordinary skill in the art will understand that for some aspects, one or more of the above steps could be eliminated, combined, or performed in a different sequence, and that other steps may be added.

FIG. 35 illustrates a flow diagram for an example method 3700 for default operation in a mobility or dithering mode with time limit, according to aspects of the present disclosure. It is understood that the steps of method 2400 may be performed in a different order than shown in FIG. 13, additional steps can be provided before, during, and after the steps, and/or some of the steps described can be replaced or eliminated in other aspects. These steps may be executed in real time or near-real time, for example as coded instructions on a processor such as processor circuit 3850 of FIG. 38, in response to inputs by a clinician or other user.

In step 3710, the method includes waiting for a user input to begin operating the surgical device in the rotational mode.

In step 3720, the method includes receiving a user input (e.g., a trigger press) to begin operation in the rotational mode.

In step 3730, the method includes operating the surgical device in the rotational mode.

In step 3740, the method includes either receiving a user input (e.g., a trigger release) to end operation in the rotational, or exceedance/expiration of a time limit since the start of the rotational mode. Such a time limit may, for example, reduce the chance of overheating the drive motors of the surgical device.

In step 3750, the method includes ceasing to operate the surgical device in the rotational mode.

In step 3760, the method includes operating the device in the default mobility or dithering mode.

In step 3770, the method includes determining an exceedance/expiration of a time limit since the start of the mobility or dithering mode.

In step 3780, the method includes ceasing to operate the surgical device in the mobility or dithering mode. Execution then returns to step 3710.

A person of ordinary skill in the art will understand that for some aspects, one or more of the above steps could be eliminated, combined, or performed in a different sequence, and that other steps may be added.

FIG. 36 is a schematic diagram of a processor circuit 3850, according to aspects of the present disclosure. The processor circuit 3850 may be implemented in the ultrasound imaging system 100, or other devices or workstations (e.g., third-party workstations, network routers, etc.), or in a cloud processor or other remote processing unit, as necessary to implement the method. As shown, the processor circuit 3850 may include a processor 3860, a memory 3864, and a communication module 3868. These elements may be in direct or indirect communication with each other, for example via one or more buses.

The processor 3860 may include a central processing unit (CPU), a digital signal processor (DSP), an ASIC, a controller, or any combination of general-purpose computing devices, reduced instruction set computing (RISC) devices, application-specific integrated circuits (ASICs), field programmable gate arrays (FPGAs), or other related logic devices, including mechanical and quantum computers. The processor 3860 may also comprise another hardware device, a firmware device, or any combination thereof configured to perform the operations described herein. The processor 3860 may also be implemented as a combination of computing devices, e.g., a combination of a DSP and a microprocessor, a plurality of microprocessors, one or more microprocessors in conjunction with a DSP core, or any other such configuration.

The memory 3864 may include a cache memory (e.g., a cache memory of the processor 3860), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an aspect, the memory 3864 includes a non-transitory computer-readable medium. The memory 3864 may store instructions 3866. The instructions 3866 may include instructions that, when executed by the processor 3860, cause the processor 3860 to perform the operations described herein. Instructions 3866 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 3868 can include any electronic circuitry and/or logic circuitry to facilitate direct or indirect communication of data between the processor circuit 3850, and other processors or devices. In that regard, the communication module 3868 can be an input/output (I/O) device. In some instances, the communication module 3868 facilitates direct or indirect communication between various elements of the processor circuit 3850 and/or the ultrasound imaging system 100. The communication module 3868 may communicate within the processor circuit 3850 through numerous methods or protocols. Serial communication protocols may include but are not limited to US SPI, I²C, RS-232, RS-485, CAN, Ethernet, ARINC 429, MODBUS, MIL-STD-1553, or any other suitable method or protocol. Parallel protocols include but are not limited to ISA, ATA, SCSI, PCI, IEEE-488, IEEE-1284, and other suitable protocols. Where appropriate, serial and parallel communications may be bridged by a UART, USART, or other appropriate subsystem.

External communication (including but not limited to software updates, firmware updates, preset sharing between the processor and central server, or readings from the ultrasound device) may be accomplished using any suitable wireless or wired communication technology, such as a cable interface such as a USB, micro USB, Lightning, or FireWire interface, Bluetooth, Wi-Fi, ZigBee, Li-Fi, or cellular data connections such as 2G/GSM, 3G/UMTS, 4G/LTE/WiMax, or 5G. For example, a Bluetooth Low Energy (BLE) radio can be used to establish connectivity with a cloud service, for transmission of data, and for receipt of software patches. The controller may be configured to communicate with a remote server, or a local device such as a laptop, tablet, or handheld device, or may include a display capable of showing status variables and other information. Information may also be transferred on physical media 610 such as a USB flash drive or memory stick.

A number of variations are possible on the examples and aspects described above. For example, the systems, devices, and techniques disclosed herein may be used to produce other cutting and/or vibration/dithering modes than those described herein. The technology described herein may be applied to cutting catheters of diverse types, whether currently in existence or hereinafter developed.

Accordingly, the logical operations making up the aspects of the technology described herein are referred to variously as operations, steps, objects, elements, components, or modules. Furthermore, it should be understood that these may be performed in any order, unless explicitly claimed otherwise or a specific order is inherently necessitated by the claim language. All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of the IVUS pullback virtual venogram system. Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” Unless otherwise noted in the claims, stated values shall be interpreted as illustrative only and shall not be taken to be limiting.

The above specification, examples and data provide a complete description of the structure and use of exemplary aspects of the cutting catheter system as defined in the claims. Although various aspects of the claimed subject matter have been described above with a certain degree of particularity, or with reference to one or more individual aspects, those skilled in the art could make numerous alterations to the disclosed aspects without departing from the spirit or scope of the claimed subject matter. Still other aspects are contemplated. It is intended that all matter contained in the above description and shown in the accompanying drawings shall be interpreted as illustrative only of particular aspects and not limiting. Changes in detail or structure may be made without departing from the basic elements of the subject matter as defined in the following claims.

What is claimed is:

1. An intraluminal cutting device, comprising:
a flexible elongate member configured to be positioned within a body lumen of a patient, wherein the flexible elongate member comprises a proximal portion and a distal portion;
a cutting tip assembly positioned at the distal portion of the flexible elongate member, wherein the cutting tip assembly comprises a cam pin, a cam slot, and a blade configured to cut tissue associated with the body lumen,
wherein the cam pin is configured to move within the cam slot along a cam path,
wherein the cam pin is configured to oscillate along only a portion of the cam path.

2. The intraluminal cutting device of claim 1,
wherein, in a first mode of operation, the cam pin is configured to move along an entirety of the cam path, and
wherein the cam pin oscillating along only the portion of the cam path is associated with a second mode of operation.

3. The intraluminal cutting device of claim 2,
wherein, in the first mode of operation, the blade is configured to provide a first type of cutting of the tissue, and
wherein, in the second mode of operation, the blade is configured to provide a second type of cutting of the tissue.

4. The intraluminal cutting device of claim 3,
wherein the first type of cutting of the tissue is associated with rotational movement of the blade, and
wherein the second type of cutting of the tissue is associated with longitudinal movement of the blade.

5. The intraluminal cutting device of claim 2,
wherein the cutting tip assembly is coupled to the flexible elongate member,
wherein, in the second mode of operation, the cam pin oscillating along only the portion of the cam path is associated with the flexible elongate member oscillating.

6. The intraluminal cutting device of claim 5, wherein the second mode of operation is associated with rotational movement of the flexible elongate member.

7. The intraluminal cutting device of claim 2, wherein the cam pin reciprocating along only the portion of the cam path is associated with a third mode of operation.

8. The intraluminal cutting device of claim 1, further comprising:
a handle positioned at the proximal portion of the flexible elongate member; and
a motor positioned within the handle, wherein the motor is configured to control the cam pin to oscillate along only a portion of the cam path.

9. The intraluminal cutting device of claim 8,
further comprising a processor circuit positioned within the handle,
wherein the processor circuit is configured to control the motor.

10. The intraluminal cutting device of claim 9,
further comprising a battery positioned within the handle,
wherein the battery is configured to power the motor and the processor circuit.

11. The intraluminal cutting device of claim 9,
wherein the cam pin oscillating along only the portion of the cam path is associated with a mode of operation,
wherein the processor circuit is configured to receive a user input associated with the mode of operation, and
wherein the processor circuit is configured to control the motor in response to the user input.

12. The intraluminal cutting device of claim 9,
wherein the cam pin oscillating along only the portion of the cam path is associated with a mode of operation,
wherein the processor circuit is configured to determine a duration for the mode of operation, and
wherein the processor circuit is configured to control the motor in response to determining the duration.

13. The intraluminal cutting device of claim 9, wherein the portion of the cam path is associated with a first region of the cam path and a second region of the cam path.

14. The intraluminal cutting device of claim 13, wherein a first region of the cam path and a second region of the cam path are opposite to one another along the cam path.

15. The intraluminal cutting device of claim 14, wherein the processor circuit is configured to control the motor such that the cam pin alternates between:
oscillating along the first region of the cam path; and
oscillating along the second region of the cam path.

16. An intraluminal cutting device, comprising:
a flexible elongate member configured to be positioned within a body lumen of a patient, wherein the flexible elongate member comprises a proximal portion, a distal portion, and a lumen configured to receive a medical device;
a handle positioned at the proximal portion of the flexible elongate member, wherein the handle comprises a motor, a processor circuit, and a battery; and
a cutting tip assembly coupled to the flexible elongate member, wherein the cutting tip assembly comprises a cam pin, a cam slot, and a blade configured to cut tissue associated with the body lumen,
wherein the processor circuit is configured to control the motor to cause the cam pin to move within the cam slot along a cam path,
wherein the cam pin is configured to oscillate along only a portion of the cam path such that at least one of:
the blade is configured to provide repeated longitudinal contact with the tissue; or
the flexible elongate member is configured to provide repeated movement relative to the medical device within the lumen of the flexible elongate member.
